(19) United States
(12) Patent Application Publication
(10) Pub. No.: US 2023/0181625 A1
(43) Pub. Date: Jun. 15, 2023

(54) METHOD AND COMPOSITION FOR TREATING INFECTIOUS CONJUNCTIVITIS

(71) Applicant: Harrow IP, LLC, Nashville, TN (US)

(72) Inventor: Dennis Saadeh, Nashville, TN (US)

(21) Appl. No.: 17/550,802

(22) Filed: Dec. 14, 2021

(51) Int. Cl. A61K 31/79; A61P 27/02; A61K 9/00; A61K 47/10

(52) U.S. Cl. CPC A61K 31/79; A61P 27/02; A61K 9/0048; A61K 47/10

(57) ABSTRACT

The present invention relates generally to a method of treating infectious conjunctivitis by administering to an infected eye of a subject a composition comprising povidone-iodine and an excipient that reduces the ocular toxicity of the composition, wherein the composition does not include a steroidal anti-inflammatory drug or a non-steroidal anti-inflammatory drug.

### FIELD OF THE INVENTION

The present invention involves a method and composition for treating infectious conjunctivitis. Accordingly, the present invention involves the fields of pharmacy, medicine, and chemistry.

### BACKGROUND

Povidone-iodine (PVP-I) is known for its anti-microbial qualities (Saggers, B. and Stewart, G., 1964). It is generally available in most pharmacies and is widely used as an antiseptic. There are also no indications of bacterial resistance to PVP-I, making it an especially important tool for fighting infectious disease. However, in ophthalmic formulations, PVP-I has been disfavored due to observed ocular intolerability at high concentrations. Furthermore, conflicting reports exist in the literature regarding PVP-I's efficacy in treating viral conjunctivitis. Some solutions to this problem have included use of PVP-I in formulations containing steroidal anti-inflammatory drugs and non-steroidal anti-inflammatory drugs to improve ocular tolerability. What is needed is a method of treating infectious conjunctivitis by administering a solution comprising PVP-I to the infected eye of a subject that does not exhibit high ocular toxicity or use steroidal or non-steroidal anti-inflammatory drugs.

### SUMMARY OF THE INVENTION

Among the various aspects of the invention is a composition useful for the treatment of infectious conjunctivitis. The composition includes PVP-I as an active ingredient, and is preferably both efficacious in the treatment of infectious conjunctivitis and shows no major differences in ocular tolerability when compared to a placebo control. The composition further includes an excipient selected from polyvinylpyrrolidone (PVP), glycerin, or a combination thereof. The excipient may reduce the ocular tolerability of the composition to a level statistically similar to a placebo control on day 9 of a Draize rabbit eye test. Additionally, the composition does not include a steroidal anti-inflammatory drug nor a non-steroidal anti-inflammatory drug. The ocular tolerability of the composition may be comparable to that of a placebo control without the use of steroidal anti-inflammatory drugs or non-steroidal anti-inflammatory drugs.

Another aspect of the invention is a method for the treatment of infectious conjunctivitis using the composition described herein. The method includes administering to an infected eye of a subject a composition comprising a therapeutically effective amount of a composition that may include PVP-I as an active ingredient. This composition may also include an excipient, which may reduce the ocular tolerability of the composition to a level statistically similar to a placebo control on day 9 of a Draize rabbit eye test. The method may not include use of a steroidal anti-inflammatory drug or a non-steroidal anti-inflammatory drug.

### BRIEF DESCRIPTION OF THE FIGURES

FIG. 1 shows the results of a Draize ocular toxicity test performed in rabbits. Formulas 1A-1C included a biguadine polymer, formula 1D included a composition of the present disclosure, and formula 1E included a vehicle control.

FIG. 2 shows the results of an Ad5 viral titer study to measure viral replication. Formulas 1A-1C included a biguadine polymer, formula 1D included a composition of the present disclosure, and formula 1E included a vehicle control.

FIG. 3 shows the results of an Ad5 positive cultures per total study to measure viral replication. Formulas 1A-1C included a biguadine polymer, formula 1D included a composition of the present disclosure, and formula 1E included a vehicle control.

FIG. 4 shows the results of an Ad5 duration of shedding study to determine the length of infection. Formulas 1A-1C included a biguadine polymer, formula 1D included a composition of the present disclosure, and formula 1E included a vehicle control.

### DETAILED DESCRIPTION

Before the present invention is disclosed and described, it is to be understood that this invention is not limited to the particular methods, compositions, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, etc. This same principle applies to ranges reciting only one numerical value as a minimum or a maximum. Furthermore, such an interpretation should apply regardless of the breadth of the range or the characteristics being described.

As used herein, the term “about” is used to provide flexibility to a numerical range endpoint by providing that a given value may be “a little above” or “a little below” the endpoint. For example, the endpoint may be within 10%, 8%, 5%, 3%, 2%, or 1% of the listed value. Further, for the sake of convenience and brevity, a numerical range of “about 50 mg/mL to about 80 mg/mL” should also be understood to provide support for the range of “50 mg/mL to 80 mg/m L.” The endpoint may also be based on the variability allowed by an appropriate regulatory body, such as the FDA, USP, etc.

In this disclosure, “comprises,” “comprising,” “containing,” and “having” and the like can have the meaning ascribed to them in U.S. Patent Law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. patent law. “Consisting essentially of” or “consists essentially of” have the meaning generally ascribed to them by U.S. patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. In this specification when using an open ended term, like “comprising” or “including,” it is understood that direct support should be afforded also to “consisting essentially of” language as well as “consisting of” language as if stated explicitly and vice versa.

As used herein, “subject” refers to a mammal. Examples of subjects include humans, and may also include other animals such as horses, pigs, cattle, dogs, cats, rabbits, and aquatic mammals. In one aspect, the subject can be a human.

As used herein, “ocular tolerability” and “ocular toxicity” refer to the degree to which overt adverse effects to an eye of a subject can be tolerated by the subject or produce measurable toxicity, respectively. Ocular tolerability and ocular toxicity are inversely related. Ocular tolerability or ocular toxicity may be measured in humans or other suitable subjects. For instance, ocular tolerability may be measured using the Draize scale. The Draize scale method involves instillation 100 μL of a test liquid into the lower conjunctival sac and cornea of one eye of a non-human subject (typically a rabbit), followed by a saline rinse. The other eye of the subject receives no liquid and serves as a control to compare against the treated eye. Observations of corneal opacity, conjunctival hyperemia, chemosis, ocular discharge, iris abnormalities, and other criteria are recorded at predefined intervals. The recorded symptoms are then developed into a “maximum average score.” Results of the Draize test may be used to describe the test liquid as non-irritating, practically non-irritating, minimally irritating, mildly irritating, moderately irritating, severely irritating, extremely irritating, or maximally irritating. Draize testing is the most commonly-performed test to measure ocular tolerability.

Other methods to measure ocular tolerability include in vivo tests such as low-volume eye-irritation tests (LVET). LVET differs from Draize testing in that lower volumes of the test substance (10 μL) are injected. However, animal subjects are still used for LVET. Further, if no irritancy occurs at the low volume in LVET testing, the standard procedure is to repeat the test with increased volume. Ex vivo tests for ocular toxicity include isolated/enucleated organ/organotypic methods; however, such tests cannot predict the systemic effects of substances and can only be used for short-term assessment periods. In vitro tests also exist to measure ocular toxicity, such as cytotoxicity assessments, corneal epithelial models, and corneal equivalents.

Another method to measure ocular tolerability is the Comparison of Ophthalmic Medication for Ocular Tolerability, or COMTOL. COMTOL is a questionnaire that patients complete after administration of an ophthalmic medication. Additional methods of measuring ocular tolerability are well-known in the art.

On the other hand, ocular toxicity may be measured in several ways, depending on the type of toxicity expected. For example, ocular toxicity may be measured by staining the corneal surface to measure damage to epithelium, or by measuring conjunctival hyperemia. Additional methods of measuring ocular toxicity are well-known in the art.

The terms “treat,” “treating,” or “treatment” as used herein, refer to both therapeutic treatment and prophylactic or preventative measures, wherein the object is to prevent or slow down (lessen) an undesired physiological change or disease/disorder. Beneficial or desired clinical results include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented.

### I. Composition for Treating Infectious Conjunctivitis.

Described herein is a composition for treating infectious conjunctivitis comprising PVP-I and an excipient selected from PVP, glycerin, or a combination thereof. The composition may also include one or more additional excipient, including but not limited to a pharmaceutically acceptable carrier, a preservative, a pH adjusting agent, a surfactant, a stabilizer, or any combination thereof. In some embodiments, the composition is an aqueous solution. Compositions of the present disclosure are typically ocular formulations effective for treating infectious conjunctivitis in a subject in need thereof, preferably with a statistically similar level of ocular toxicity as compared to a placebo control. As used herein, a “placebo control” is a composition identical in structure to the composition it is being evaluated against except the placebo control lacks PVP-I.

In some embodiments, the composition includes a PVP-I concentration of about 0.6% (w/w) to about 1.25%. For example, the composition may include a PVP-I concentration of about 0.6%, 0.65%, 0.7%, 0.75%, 0.8%, 0.85%, 0.9%, 0.95%, 1%, 1.05%, 1.1%, 1.15%, 1.2%, or 1.25% (w/w). In some examples, the concentration of PVP-I may be about 0.6% to about 1%, about 0.6% to about 0.9%, or about 0.6% to about 0.8% (w/w). In some examples, the concentration of PVP-I may be about 0.7% to about 1%, or about 0.7% to about 0.9% (w/w). In some examples, the concentration of PVP-I may be about 0.8% to about 1.2%, about 0.8% to about 1.1%, or about 0.8% to about 1.0% (w/w). In some examples, the concentration of PVP-I may be about 0.6% to about 0.7%, about 0.7% to about 0.8%, about 0.8% to about 0.9%, about 0.9% to about 1%, about 1% to about 1.1%, about 1.1% to about 1.2%, or about 1.2% to about 1.25% (w/w). In one exemplary embodiment, the concentration of PVP-I is at about 0.8% (w/w). In certain embodiments, the composition may include PVP-I as the sole active ingredient.

In some embodiments, the composition may comprise an amount of PVP, glycerin, or a combination thereof that improves ocular tolerability to the PVP-I in the composition. In some embodiments the concentration of each excipient in the composition may be about 1.0% to about 2.5% (w/w). For example, the concentration of each excipient may be about 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or about 2.5% (w/w). In other examples, the concentration of each excipient may be about 1.0% to about 1.1%, about 1.1% to about 1.2%, about 1.2% to about 1.3%, about 1.3% to about 1.4%, about 1.4% to about 1.5%, about 1.5% to about 1.6%, about 1.6% to about 1.7%, about 1.7% to about 1.8%, about 1.8% to about 1.9%, about 1.9% to about 2.0%, about 2.0% to about 2.1%, about 2.1% to about 2.2%, about 2.2% to about 2.3%, about 2.3% to about 2.4%, or about 2.4% to about 2.5% (w/w). Ocular tolerability may be measured by any suitable method known in the art. For instance, ocular tolerability may be measured in human subjects during a clinical trial. Alternatively, ocular tolerability may be measured in a suitable animal model, such as the Draize rabbit eye test detailed in the examples. The amount of the excipient(s) in a composition of the present disclosure can be shown to improve ocular tolerability by comparison to a composition that lacks the excipient(s) but is otherwise identical in structure. The improvement in ocular tolerability may be a reduction in ocular toxicity after administration for 2 or more consecutive days (e.g., 2, 3, 4, 5, 6, 7, 8, 9, 10, 11, 12, 13, 14, or more days), wherein the amount of the reduction is about 10%, about 20%, about 30%, about 40%, about 50%, about 60%, about 70%, about 80%, about 90% or more. Alternatively, or in addition, the amount of the excipient(s) in a composition of the present disclosure can be shown to improve ocular tolerability when the composition comprising the excipient(s) reduces ocular toxicity to a level statistically similar to a placebo control. In exemplary embodiments, an improvement in ocular tolerability is measured after administration for at least 2 consecutive days (e.g., 2, 3, 4, 5, 6, 7, 8, 9, 10, 11, 12, 13, 14, or more days), more preferably after administration for at least 7 consecutive days or 9 days, even more preferably after administration for at least 14 consecutive days

In some embodiments, the excipient may be PVP. The PVP may have a molecular weight of about 25,000 to about 40,000. In some aspects, the PVP may have a molecular weight of about 25,000 to about 30,000, about 30,000 to about 35,000, or about 35,000 to about 40,000. In some additional aspects, the PVP may have a molecular weight of about 25,000, 30,000, 35,000, or about 40,000. In some examples, the PVP may have a molecular weight of about 30,000.

The concentration of PVP in the composition may be about 0.5% to about 2.5%, or more preferably about 1.0% to about 2.5% (w/w). For example, the concentration of PVP may be about 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or 2.5% (w/w). In some examples, the concentration of PVP may be about 1.0% to about 2.2%, about 1.0% to about 2.0%, about 1.0% to about 1.8%, or about 1.0% to about 1.5%. In some examples, the concentration of PVP may be about 0.7% to about 1.3%, about 0.8% to about 1.2%, or about 0.9% to about 1.1%. In some examples, the concentration of PVP may be about 0.9% to about 1.0%, about 1.0% to about 1.1%, about 1.1% to about 1.2%, about 1.2% to about 1.3%, about 1.3% to about 1.4%, about 1.4% to about 1.5%, about 1.5% to about 1.6%, about 1.6% to about 1.7%, about 1.7% to about 1.8%, about 1.8% to about 1.9%, about 1.9% to about 2.0%, about 2.0% to about 2.1%, about 2.1% to about 2.2%, about 2.2% to about 2.3%, about 2.3% to about 2.4%, or about 2.4% to about 2.5% (w/w). In some examples, the concentration of PVP is about 1%. In some examples, the PVP is PVP-k30.

In some embodiments, the excipient may be glycerin. The concentration of glycerin in the composition may be about 1.0% to about 2.5% (w/w). For example, the concentration of glycerin may be about 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or 2.5% (w/w). In some examples, the concentration of PVP may be about 1.0% to about 2.2%, about 1.0% to about 2.0%, about 1.0% to about 1.8%, or about 1.0% to about 1.5%. In some examples, the concentration of PVP may be about 1.3% to about 2.5%, about 1.5% to about 2.5%, about 1.7% to about 2.5%, or about 2.0% to about 2.5%. In other examples, the concentration of glycerin may be about 1.0% to about 1.1%, about 1.1 to about 1.2%, about 1.2% to about 1.3%, about 1.3% to about 1.4%, about 1.4% to about 1.5%, about 1.5% to about 1.6%, about 1.6% to about 1.7%, about 1.7% to about 1.8%, about 1.8% to about 1.9%, about 1.9% to about 2.0%, about 2.0% to about 2.1%, about 2.1% to about 2.2%, about 2.2% to about 2.3%, about 2.3% to about 2.4%, or about 2.4% to about 2.5% (w/w). In some embodiments, the concentration of glycerin is about 1.25% (w/w).

In some embodiments, the excipient may be glycerin and PVP. The concentration of glycerin may be about 1.0% and about 2.5% (w/w) and the concentration of PVP in the composition may be about 0.5% and about 2.5% (w/w). In various examples, the concentration of glycerin and PVP may be as described in the preceding paragraphs. In some embodiments, the concentration of PVP is about 0.8% to about 1.2% (w/w) and the concentration of glycerin is about 1.0% to about 1.5% (w/w). In some embodiments, the concentration of PVP is about 1% to about 1.5% (ww/w) and the concentration of glycerin is about 1.0% to about 1.5% (w/w). In some embodiments, the concentration of PVP is about 1% (w/w) and the concentration of glycerin is about 1.25% (w/w).

The composition described herein does not include amounts of steroidal anti-inflammatory drug(s) or non-steroidal anti-inflammatory drug(s) to improve the ocular tolerability of the composition. Examples of steroidal anti-inflammatory drugs include betamethasone, corticosteroids, dexamethasone, fluromethalone, hydrocortisone acetate, prednisolone, rimexolone, loteprednol etabonate, and other steroidal anti-inflammatory drugs known in the art. Examples of non-steroidal anti-inflammatory drugs include bromfenac, diclofenac, flurbiprofen sodium, ketorolac tromethamine, nepafenac, and other non-steroidal anti-inflammatory drugs known in the art. The composition described herein also does not include amounts of N-acetylcysteine that improve ocular tolerability of the composition. Accordingly, in certain embodiments, compositions of the present disclosure may consist essentially of (a) PVP-I, (b) an excipient selected from PVP, glycerin, or a combination thereof, and (c) one or more additional active ingredient or excipient, with the proviso that the components of (c) do not materially affect the ocular tolerability of the composition. In still other embodiments, compositions of the present disclosure may consist essentially of (a) PVP-I, (b) an excipient selected from PVP, glycerin, or a combination thereof, and (c) one or more additional excipient, each independently selected from the group consisting of a pH adjusting agent, a preservative, a stabilizer, and a surfactant, with the proviso that the components of (c) do not materially affect the ocular tolerability of the composition. Methods for determining whether a drug or an excipient improves ocular tolerability of a composition are described above. In exemplar embodiments, ocular tolerability is evaluated on day 9 of a Draize rabbit eye test.

In some embodiments, the composition may include a demulcent. In some aspects, the demulcent may be polyethylene glycol, polysorbates (such as Polysorbate 80), polyvinyl alcohol, propylene glycol, glycerin, cellulose derivatives (e.g., carboxymethylcellulose sodium, hydroxyethyl cellulose, hypromellose, and methylcellulose), Dextran 70, gelatin, and other demulcents known in the art and combinations thereof. When the demulcent is polyethylene glycol, the polyethylene glycol may have a molecular weight of about 300 (PEG-300) to about 400 (PEG-400).

The demulcent may have a concentration in the composition of about 0.1% (w/w) to about 5% (w/w). In some embodiments, the demulcent may have a concentration in the composition of between about 0.1% (w/w) to about 0.5% (w/w), about 0.5% (w/w) to about 1% (w/w), about 1% (w/w) to about 1.5% (w/w), about 1.5% (w/w) to about 2% (w/w), about 2% (w/w) to about 2.5% (w/w), about 2.5% (w/w) to about 3% (w/w), about 3% (w/w) to about 3.5% (w/w), about 3.5% (w/w) to about 4% (w/w), about 4% (w/w) to about 4.5% (w/w), or about 4.5% (w/w) to about 5% (w/w). In some additional embodiments, the demulcent may have a concentration in the composition of between about 0.1% (w/w) to about 4.5% (w/w), 0.1% (w/w) to about 4% (w/w), about 0.1% (w/w) to about 3.5% (w/w), about 0.1% (w/w) to about 3% (w/w), about 0.1% (w/w) to about 2.5% (w/w), about 0.1% (w/w) to about 2% (w/w), about 0.1% (w/w) to about 1.5% (w/w), about 0.1% (w/w) to about 1% (w/w), about 0.5% (w/w) to about 5% (w/w), about 1% (w/w) to about 5% (w/w), about 1.5% (w/w) to about 5% (w/w), about 2% (w/w) to about 5% (w/w), about 2.5% (w/w) to about 5% (w/w), about 3% (w/w) to about 5% (w/w), about 3.5% (w/w) to about 5% (w/w), or about 4% (w/w). In still further embodiments, the demulcent may have a concentration in the composition of about 0.1% (w/w), 0.2% (w/w), 0.3% (w/w), 0.4% (w/w) 0.5% (w/w), 0.6% (w/w), 0.7% (w/w), 0.8% (w/w), 0.9% (w/w), 1% (w/w), 1.5% (w/w), 2% (w/w), 2.5% (w/w), 3% (w/w), 3.5% (w/w), 4% (w/w), 4.5% (w/w), or about 5% (w/w). In one example, the demulcent is PEG-400 and the PEG-400 has a concentration in the composition of about 4% (w/w).

In order to maintain the stability of the composition, the pH of the composition must be controlled. The pH of the composition may be about 4.2 to about 4.5. In some examples, the pH of the composition may be about 4.2, 4.3, 4.4 or 4.5. In some embodiments, the composition may include a pH adjusting agent (e.g., an acid, a base, a buffer) to achieve or maintain a desired pH. The pH adjusting agent may include dibasic sodium phosphate, boric acid, citric acid, or a combination thereof.

In some embodiments, the pH adjusting agent may include citric acid. In some aspects, the citric acid may have a concentration in the composition of between about 0.1% (w/w) to about 1% (w/w). In some additional aspects, the concentration of the citric acid may be between about 0.1% (w/w) to about 0.7% (w/w), about 0.1% (w/w) to about 0.5% (w/w), or about 0.1% (w/w) to about 0.3% (w/w). In further additional aspects, the citric acid may have a concentration in the composition of between about 0.1% (w/w) to about 0.2% (w/w), about 0.2% (w/w) to about 0.3% (w/w), about 0.3% (w/w) to about 0.4% (w/w), about 0.4% (w/w) to about 0.5% (w/w), about 0.5% (w/w) to about 0.6% (w/w), about 0.6% (w/w) to about 0.7% (w/w), about 0.7% (w/w) to about 0.8% (w/w), about 0.8% (w/w) to about 0.9% (w/w), or about 0.9% (w/w) to about 1% (w/w). In still further aspects, the citric acid may have a concentration in the composition of about 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), or about 1% (w/w). In one example, the citric acid has a concentration in the composition of about 0.2% (w/w).

In some embodiments, the pH adjusting agent may include dibasic sodium phosphate. In some aspects, the dibasic sodium phosphate may have a concentration in the composition of between about 0.3% (w/w) to about 1.5% (w/w). In some additional aspects, the dibasic sodium phosphate may have a concentration in the composition of between about 0.3% (w/w) to about 1% (w/w), or between about 0.3% to about 0.5% (w/w). In further additional aspects, the dibasic sodium phosphate may have a concentration in the composition of between about 0.3% (w/w) to about 0.4% (w/w), about 0.4% (w/w) to about 0.5% (w/w), about 0.5% (w/w) to about 0.6% (w/w) about 0.6% (w/w) to about 0.7% (w/w), about 0.7% (w/w) to about 0.8% (w/w), about 0.8% (w/w) to about 0.9% (w/w), about 0.9% (w/w) to about 1% (w/w), about 1% (w/w) to about 1.1% (w/w), about 1.1% (w/w) to about 1.2% (w/w), about 1.2% (w/w) to about 1.3% (w/w), about 1.3% (w/w) to about 1.4% (w/w), or about 1.4% (w/w) to about 1.5% (w/w). In still further aspects, the dibasic sodium phosphate may have a concentration I the composition of about 0.3% (w/w), 0.4% (w/w), 0.5% (w/w), 0.6% (w/w), 0.7% (w/w), 0.8% (w/w), 0.9% (w/w), 1% (w/w), 1.1% (w/w), 1.2% (w/w), 1.3% (w/w), 1.4% (w/w), or about 1.5% (w/w). In one example, the dibasic sodium phosphate has a concentration in the composition of about 0.3% (w/w).

In some embodiments, the pH adjusting agent may be a combination of citric acid and dibasic sodium phosphate. In one example, the pH adjusting agent is a combination of citric acid and dibasic sodium phosphate, wherein the citric acid has a concentration in the composition of about 0.2% (w/w) and the dibasic sodium phosphate has a concentration in the composition of about 0.3% (w/w).

In some embodiments, the composition includes one or more preservatives. The preservative may include benzalkonium chloride, thimerosal, chlorobutanol, methyl paraben, propylparaben, phenyl ethyl alcohol, EDTA, sorbic acid, polyquarternium 1, or a combination thereof. In some embodiments, the preservative may have a concentration between about 0.001% and about 0.1% in the composition (w/w). For example, the concentration of the preservative may be about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, or about 0.1% (w/w). In other examples, the concentration of the preservative may be about 0.001% to about 0.002%, 0.002% to about 0.003%, about 0.003% to about 0.004%, about 0.004% to about 0.005%, about 0.006% to about 0.007%, about 0.007% to about 0.008%, about 0.008% to about 0.009%, about 0.009% to about 0.01%, about 0.01% to about 0.02%, about 0.02% to about 0.03%, about 0.03% to about 0.04% about 0.05% to about 0.06%, about 0.06% to about 0.07%, about 0.07% to about 0.08%, about 0.08% to about 0.09%, or about 0.09% to about 0.1% (w/w).

In some embodiments, the preservative may be a combination of benzalkonium chloride and EDTA. The concentration of benzalkonium chloride and the concentration of EDTA in the composition may each be about 0.001% to about 0.1% (w/w). For example, the concentration of benzalkonium chloride may be about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, or about 0.1% (w/w), and the concentration of EDTA may be about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, or about 0.1% (w/w). In other examples, the concentration of benzalkonium chloride may be about 0.001% to about 0.002%, 0.002% to about 0.003%, about 0.003% to about 0.004%, about 0.004% to about 0.005%, about 0.006% to about 0.007%, about 0.007% to about 0.008%, about 0.008% to about 0.009%, about 0.009% to about 0.01%, about 0.01% to about 0.02%, about 0.02% to about 0.03%, about 0.03% to about 0.04% about 0.05% to about 0.06%, about 0.06% to about 0.07%, about 0.07% to about 0.08%, about 0.08% to about 0.09%, or about 0.09% to about 0.1% (w/w), and the concentration of the EDTA may be about 0.001% to about 0.002%, 0.002% to about 0.003%, about 0.003% to about 0.004%, about 0.004% to about 0.005%, about 0.006% to about 0.007%, about 0.007% to about 0.008%, about 0.008% to about 0.009%, about 0.009% to about 0.01%, about 0.01% to about 0.02%, about 0.02% to about 0.03%, about 0.03% to about 0.04% about 0.05% to about 0.06%, about 0.06% to about 0.07%, about 0.07% to about 0.08%, about 0.08% to about 0.09%, or about 0.09% to about 0.1% (w/w). For example, the composition may include benzalkonium chloride at a concentration of about 0.003% (w/w) and EDTA at a concentration of about 0.1% (w/w).

The composition may include a surfactant. Generally, non-ionic surfactants are preferred because they do not bind with the PVP-I. In some embodiments, the surfactant may include polyoxyethylene sorbitan monolaurate, polyoxyethylene sorbitan monopalmitate, polyoxyethylene sorbitan monostearate, polyoxyethylene sorbitan monooleate, Polysorbate 20, Polysorbate 60, Polysorbate 80, poly(oxyethylene-co-oxypropylene) block copolymer, poloxamers, Pluronic F-68, Pluronic F-84, Pluronic F-127, Pluronic P-103, cyclodextrin, nonoxynol-9, nonylphenoxy poly(ethyleneoxy)ethanol, Igepal Co-630, tyloxapol, or any combination thereof. The concentration of the surfactant may be between about 0.01% to 2% (w/w). For example, the concentration of the surfactant in the composition may be about 0.01%, 0.02%, 0.03%, 0.04%, 0.05%, 0.06%, 0.07%, 0.08%, 0.09%, 0.1%, 0.2%, 0.3%, 0.4%, 0.5% 0.6%, 0.7%, 0.8%, 0.9%, 1%, 1.1%, 1.2%, 1.3%, 1.4%, 1.5%, 1.6%, 1.7%, 1.8%, 1.9%, or 2% (w/w). In another example, the concentration of the surfactant may be about 0.01% to about 0.02%, about 0.02% to about 0.03%, about 0.03% to about 0.04%, about 0.04% to about 0.05%, about 0.05% to about 0.06%, about 0.06% to about 0.07%, about 0.07% to about 0.08%, about 0.08% to about 0.09%, about 0.09% to about 0.1%, about 0.1% to about 0.2%, about 0.2% to about 0.3%, about 0.3% to about 0.4% about 0.4% to about 0.5%, about 0.5% to about 0.6%, about 0.6% to about 0.7%, about 0.7% to about 0.8%, about 0.8% to about 0.9%, about 0.9% to about 1%, about 1% to about 1.1%, about 1.1% to about 1.2%, about 1.2% to about 1.3%, about 1.3% to about 1.4%, about 1.4% to about 1.5%, about 1.5% to about 1.6%, about 1.6% to about 1.7%, about 1.7% to about 1.8%, about 1.8% to about 1.9%, or about 1.9% to about 2% (w/w). In one example, the surfactant is tyloxapol and has a concentration of about 0.02% to about 0.5% (w/w). In another example, the surfactant is tyloxapol at a concentration of about 0.3% (w/w).

The composition may further include a stabilizer. In some embodiments, the stabilizer may include calcium chloride, magnesium chloride, sodium chloride or any other chloride salt. The stabilizer may have a concentration in the composition of about 0.1% to about 0.9%. For example, the stabilizer may have a concentration in the composition of about 0.1% (w/w), 0.2% (w/w), 0.3% (w/w), 0.4% (w/w), 0.5% (w/w), 0.6% (w/w), 0.7% (w/w), 0.8% (w/w), or about 0.9% (w/w). In one embodiment, the stabilizer is sodium chloride at a concentration of about 0.2% (w/w).

The composition may further comprise water. In some embodiments, the composition is an aqueous solution. In some embodiments, the composition may be a viscous solution. In some aspects, the composition may have a viscosity of about 1 cP to about 100 cP. In some additional aspects, the composition may have a viscosity of about 1 cP to about 5 cP, about 5 cP to about 10 cP, about 10 cP to about 20 cP, about 20 cP to about 30 cP, about 30 cP to about 40 cP, about 40 cP to about 50 cP, about 50 cP to about 60 cP, about 60 cP to about 70 cP, about 70 cP to about 80 cP, about 80 cP to about 90 cP, or about 90 cP to about 100 cP. In further aspects, the composition may have a viscosity of about 1 cP to about 90 cP, about 1 cP to about 80 cP, about 1 cP to about 70 cP, about 1 cP to about 60 cP, about 1 cP to about 50 cP, about 1 cP to about 40 cP, about 1 cP to about 30 cP, about 1 cP to about 20 cP, about 1 cP to about 10 cP, about 5 cP to about 100 cP, about 10 cP to about 100 cP, about 20 cP to about 100 cP, about 30 cP to about 100 cP, about 40 cP to about 100 cP, about 50 cP to about 100 cP, about 60 cP to about 100 cP, about 70 cP to about 100 cP, or about 90 cP to about 100 cP. In still further aspects, the composition may have a viscosity of about 1 cP, 5 cP, 10 cP, 20 cP, 30 cP, 40 cP, 50 cP, 60 cP, 70 cP, 80 cP, 90 cP, or about 100 cP.

In an exemplary embodiment, the composition comprises about 0.6% (w/w) to about 1.25% (w/w) PVP-I, about 1% (w/w) to about 2% (w/w) glycerin, about 1% (w/w) to about 2% (w/w) PVP, about 0.1% (w/w) to about 1% (w/w) citric acid, about 0.3% (w/w) to about 1.5% (w/w) dibasic sodium phosphate, about 0.001% (w/w) to about 0.1% (w/w) benzalkonium chloride, about 0.1% (w/w) to about 0.9% (w/w) sodium chloride, about 4% (w/w) PEG-400, and qs ad water for injection, wherein the composition has a pH of between about 4.2 to about 4.5. In another exemplary embodiment, the composition comprises about 0.8% (w/w) PVP-I, about 1.25% (w/w) glycerin, about 1% (w/w) PVP, about 0.2% (w/w) citric acid, about 0.3% (w/w) dibasic sodium phosphate, about 0.003% (w/w) benzalkonium chloride, about 0.2% (w/w) sodium chloride, about 4% (w/w) PEG-400, and qs ad water for injection, wherein the composition has a pH of about 4.2 to about 4.5.

In one example, the composition is prepared by first providing water for injection, then adding and dissolving sequentially the pH adjusting agent, the stabilizer, the excipient, the demulcent, the PVP-I, and the preservative. Next, the pH may be measured and adjusted. Finally, water for injection is added until a desired final volume is reached.

### II. Method of Treating Infectious Conjunctivitis

Described herein is a method of treating infectious conjunctivitis in a subject in need thereof. The method comprises administering to an infected eye of the subject a therapeutically effective amount of a composition of Section I. In some embodiments, the method comprises administering to an infected eye of the subject a therapeutically effective amount of a composition comprising about 0.6% to 1.25% (w/w) povidone-iodine and an amount of an excipient selected from polyvinylpyrrolidone, glycerin, or a combination thereof, wherein the amount of the excipient reduces ocular toxicity of the povidone-iodine to a level statistically similar to a placebo control. In some embodiments, the method comprises administering to an infected eye of the subject a therapeutically effective amount of a composition comprising about 0.6 to 1.25% (w/w) povidone-iodine; about 1 to 2% (w/w) polyvinylpyrrolidone; about 1 to 2% (w/w) glycerin, wherein the composition produces a statistically similar level of ocular toxicity as compared to a placebo control. In some embodiments, the method may comprise administering the composition to a non-infected eye of the subject, for instance, to prevent infection of the non-infected eye while the infected eye is being treated. Additional aspects of suitable compositions are described in Section I and incorporated herein by reference.

In some embodiments, the type of infectious conjunctivitis may be bacterial conjunctivitis. For example, the method may be used to treat bacterial conjunctivitis caused by Staphylococcus aureus, Coagulase-negative Staphylococcus, Staphylococcus epidermidis, Escherichia coli, Pseudomonas aeruginosa, Serratia marcescens, Streptococcus pneumoniae, Haemophilus influenzae, or other bacterial species.

In some embodiments, the type of infectious conjunctivitis may be viral conjunctivitis. For example, the method may be used to treat viral conjunctivitis caused by an adenovirus, a herpes simplex virus, an enterovirus, or other virus species.

Methods described herein comprise administering a therapeutically effective amount of a composition of Section I. As used herein, the term “therapeutically effective amount” means an amount that leads to measurable and beneficial effects for the subject administered the substance, i.e., significant efficacy. In some embodiments, a method of the present disclosure may result in a measurable improvement in a symptom or a clinical sign of infectious conjunctivitis. Non-limiting symptoms and clinical signs of infectious conjunctivitis include conjunctival hyperemia, discharge, ocular irritation, ocular inflammation, a positive microbial or viral test of an ocular sample (e.g., fluid, discharge, scraping, etc.), and the like. The therapeutically effective amount administered can be determined using standard techniques in the art and may be by influenced by the circumstances surrounding the case, e.g. type of conjunctivitis, severity of disease, etc. The therapeutically effective amount can be administered over any suitable number of doses, for instance, as 1, 2, 3, 4, or more doses per day, wherein a “dose” is a specified number of drops per eye. In one example, the therapeutically effective amount is administered in multiple doses, such as 4 individual doses per eye per day. In another example, the therapeutically effective amount is administered in multiple doses, such as 2 doses per eye per day. In still another example, one dose is considered one drop of the composition.

In some embodiments, a method of the present disclosure may result in a decrease in viral or microbial replication measured in a sample of ocular fluid obtained from the subject after treatment for at least 2 days (e.g., 2, 3, 4, 5, 6, 7, 8, 9, 10, 11, 12, 13, or 14 or more days), as compared to a sample of ocular fluid obtained from the subject prior to administration of the composition or as compared to viral or microbial replication measured in a sample of ocular fluid obtained from subjects administered a placebo control. Alternatively, or in addition, in some embodiments, the method may result in a decrease in viral or microbial titer measured in a sample of ocular fluid obtained from the subject after treatment for at least 2 days (e.g., 2, 3, 4, 5, 6, 7, 8, 9, 10, 11, 12, 13, or 14 or more days), as compared to a sample of ocular fluid obtained from the subject prior to administration of the composition or as compared to viral or microbial replication measured in a sample of ocular fluid obtained from subjects administered a placebo control. A high rate of viral or microbial replication or high viral or microbial titer generally indicates a more severe infection, and the amount of change in replication or titer is a direct measure of efficacy. In some examples, the decrease in viral or microbial titer may be a log 3 reduction in viral or microbial titer after treatment for at least 2 days (e.g., 2, 3, 4, 5, 6, 7, 8, 9, 10, 11, 12, 13, or 14 or more days). In some embodiments, the method may result in an increase in negative cultures from samples of ocular fluid obtained from the subject after treatment for at least 2 days (e.g., 2, 3, 4, 5, 6, 7, 8, 9, 10, 11, 12, 13, or 14 or more days), as compared to samples of ocular fluid obtained from the subject prior to administration of the composition or as compared to samples obtained from subjects administered a placebo control. A high percentage of positive cultures obtained over a number of consecutive days generally indicates a severe infection, and the amount of change in the percentage of negative cultures over the same number of days is a direct measure of efficacy.

In some embodiments, the method may result in a decrease in duration of viral shedding from samples of ocular fluid obtained from the subject after treatment for at least 2 days (e.g., 2, 3, 4, 5, 6, 7, 8, 9, 10, 11, 12, 13, or 14 or more days), as compared to viral shedding measured in a sample of ocular fluid obtained prior to administration of the composition or as compared to viral shedding measured in a sample of ocular fluid from subjects administered a placebo control. A high duration of viral shedding indicates a severe infection, and the amount of change in the duration of viral shedding is a direct measure of efficacy.

### III. Method of Preventing an Infection

Described herein is a method of preventing an infection in a subject. The method comprises administering to a non-infected eye of a subject an effective quantity of a composition of Section I. In some embodiments, the method comprises administering to a non-infected eye of the subject a therapeutically effective amount of a composition comprising about 0.6% to 1.25% (w/w) povidone-iodine and an amount of an excipient selected from polyvinylpyrrolidone, glycerin, or a combination thereof, wherein the amount of the excipient reduces ocular toxicity of the povidone-iodine to a level statistically similar to a placebo control. The method may be useful to prophylactically prevent infection of one or both eyes.

In some embodiments, the method may be useful to prevent infection after an ophthalmic surgery or procedure to prevent infection to the one or both eyes. In some aspects, the ophthalmic surgery or procedure may include cataract surgery, MIGS procedure, LAS IK, keratoplasty, or other ophthalmic surgeries or procedures known in the art.

### EXAMPLES

The following examples illustrate various iterations of the invention.

### Example 1—In-Vivo Rabbit Model Studies

The disclosed composition to enhance ocular tolerability and efficacy of PVP-I composition was tested in-vivo in New Zealand white rabbit specimens against adenovirus type 5 (Ad5; the Ad5/NZW rabbit ocular model). The purpose of the studies was to determine the antiviral efficacy of several concentrations and dosing regimens of the disclosed composition in the Ad5/NZW rabbit ocular model.

The composition used in the study comprised 0.8% PVP-I, 0.003% benzalkonium chloride, 0.3% tyloxapol, 0.1% dibasic sodium phosphate, 0.1% boric acid, 0.1% calcium chloride, 0.1% EDTA, 1% PVP, 1.25% glycerin, and water.

In the study, rabbits were treated according to the regimens described in Table 1. The povidone-iodine composition was compared to a biguanide polymer composition. Five rabbits were administered a vehicle containing no active ingredient eight times daily for ten days. One rabbit in the vehicle control group was ordered euthanized on Day 7 due to poor health.

TABLE 1
In-vivo Rabbit Model Treatment Regimen

| | | Treatment | n | n | Rabbit |
|---|---|---|---|---|---|
| Group | Drug | Regimen | Rabbits | Eyes | Numbers |
| 1A | 0.25% Biguanide | 4 times daily | 5 | 10 | 1-5 |
| | polymer | for 10 days | | | |
| 1B | 0.1% Biguanide | 4 times daily | 5 | 10 | 6-10 |
| | polymer | for 10 days | | | |
| 1C | 0.1% Biguanide | 8 times daily | 5 | 10 | 11-15 |
| | polymer | for 10 days | | | |
| 1D | 0.8% Povidone- | 4 times daily | 5 | 10 | 16-20 |
| | iodine | for 10 days | | | |
| 1E | Vehicle | 8 times daily | 5 | 10 | 21-25 |
| | | for 10 days | | | |

The outcome variables the study tracked included ocular tolerability (utilizing the Draize scale), Ad5 viral titers (to measure viral replication), Ad5 positive cultures per total (to measure viral replication), and Ad5 duration of shedding (to determine length of infection).

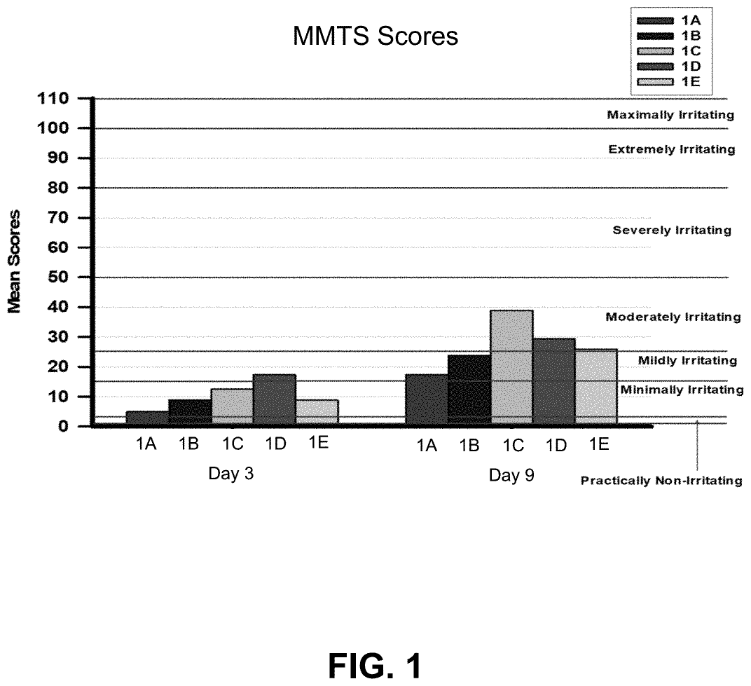

Ocular Tolerability. The results of the ocular tolerability study are shown in FIG. 1. The study showed that there are no major differences in ocular tolerability on Day 3 vs. Day 9 among the groups, including the placebo control group, wherein Day 3 refers to the third day of treatment. On Day 3, the PVP-I composition was described as “mildly irritating” according to the Draize test, while the placebo control was described as “minimally irritating.” On Day 9, the PVP-I composition and the placebo control were both described as “moderately irritating” according to the Draize test. This result indicates that the PVP-I composition showed no major differences in ocular toxicity as compared to the placebo control group.

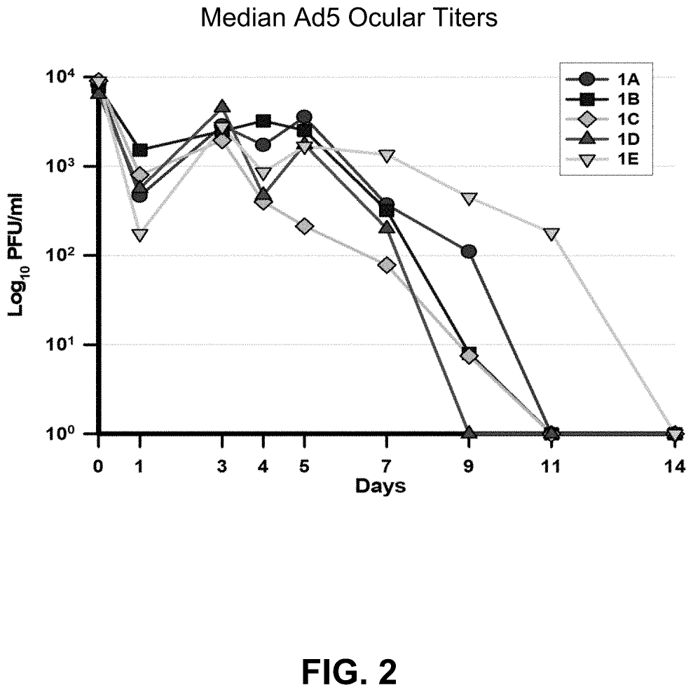

Ad5 Viral Titers. The results of the Ad5 viral titer study are shown in FIG. 2. Results of the study indicated a statistically significant reduction in viral titers (p value=0.0001) on Day 9 of the regimen. On Day 9, the median number of viral titers detected in the group receiving the PVP-I composition was zero. It was not until Day 14 that the median number of viral titers detected in the placebo control group dropped to zero. This result indicates that the PVP-I composition slowed viral replication more effectively than the placebo control.

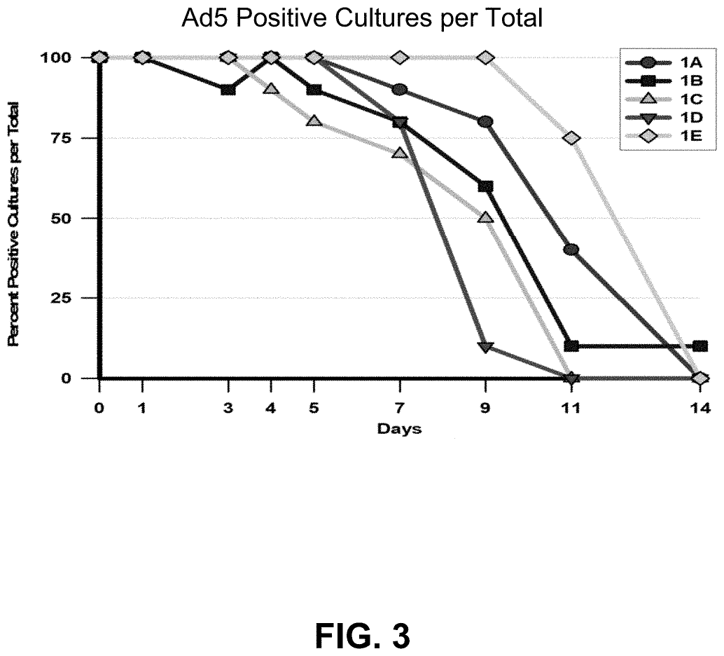

Ad5 Positive Cultures per Total. The results of the Ad5 positive cultures per total study are shown in FIG. 3. For all groups in the study, consecutive days of decreasing positive cultures began on Day 4, except for the placebo control group. For the rabbits given the 0.8% PVP-I composition, the reduction in positive cultures by Day 9 was statistically significant (p value=0.0004). On Day 11, the percent of positive cultures per total was zero for the group receiving the PVP-I composition. It was not until Day 14 that positive cultures per total reached 0% for the placebo control group. This result indicates that the PVP-I composition slowed viral replication more effectively than the placebo control.

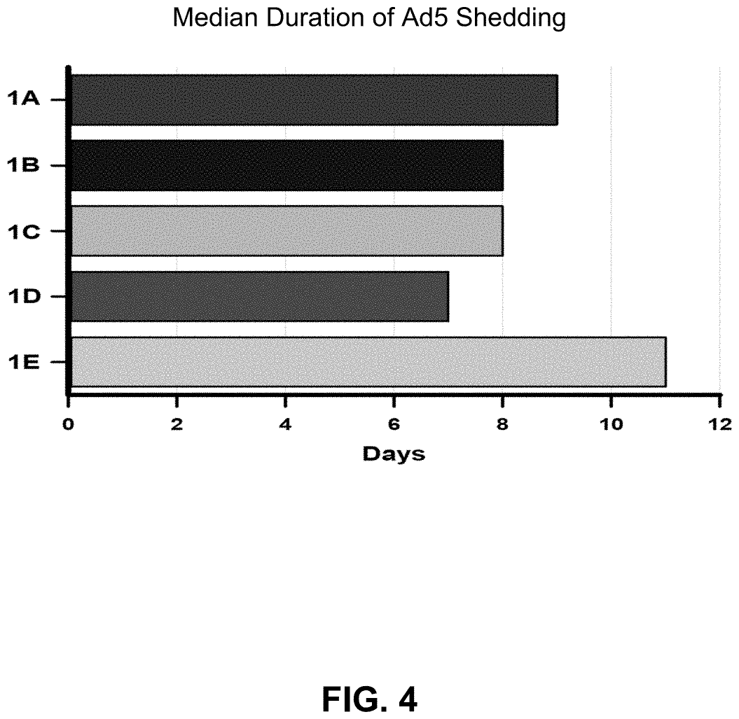

Duration of Ad5 Shedding. Results for the Ad5 shedding study are shown in FIG. 4. For the treatment group receiving the 0.8% PVP-I composition, the median duration of Ad5 shedding was about days, whereas the placebo control group showed shedding at 11 days. This indicated a statistically significant reduction in viral shedding for the group receiving the PVP-I composition (p value=0.0017). This result indicated that the PVP-I composition decreased the length of the infection more than the placebo control.

The study thus confirmed antiviral activity in vivo on Ad5/NZW rabbit ocular models and also confirmed that the composition exhibited no greater ocular toxicity than a placebo control.

### Example 2—Stability and Potency

A composition of the present disclosure was prepared according to the methods described herein having a concentration of 0.8% povidone iodine. The composition was provided to a third-party quality testing service on Jan. 20, 2021. The composition was stored at room temperature for the duration of the potency testing. The potency of the composition was tested on Feb. 2, 2021, and was tested at 30-day intervals thereafter. Table 1 shows the results of the potency testing. The composition was determined to be potent if the amount of povidone iodine in the composition was 85.0-120.0% of the label claim. Potency was determined through titration.

TABLE 1
Potency Data

| Time Point | Label Claim | Amount Found | % Label Claim | Pass/Fail |
|---|---|---|---|---|
| Baseline | 0.8% | 0.7868% | 98.3% | Pass |
| Day 30 | 0.8% | 0.7677% | 96.0% | Pass |
| Day 60 | 0.8% | 0.7611% | 95.0% | Pass |
| Day 90 | 0.8% | 0.7614% | 95.2% | Pass |
| Day 120 | 0.8% | 0.7614% | 95.2% | Pass |
| Day 150 | 0.8% | 0.7551% | 94.4% | Pass |
| Day 180 | 0.8% | 0.7233% | 90.4% | Pass |

1. A method for treating infectious conjunctivitis in a subject in need thereof, the method comprising administering to an infected eye of the subject a therapeutically effective amount of a composition consisting of:
about 0.6 to 1.25% (w/w) povidone-iodine;
about 1 to 2% (w/w) of an excipient selected from polyvinylpyrrolidone, glycerin, or a combination thereof;
optionally a preservative;
optionally a surfactant;
optionally a pH adjusting agent;
optionally a stabilizer;
optionally a demulcent; and
water,
wherein the excipient reduces ocular toxicity of the composition to a level statistically similar to a placebo control on day 9 of a Draize rabbit eye test, and wherein the composition does not contain a steroidal anti-inflammatory drug or a non-steroidal anti-inflammatory drug.

2. The method of claim 1, wherein the povidone-iodine has a concentration of about 0.8% (w/w).

3. The method of claim 1, wherein the excipient comprises polyvinylpyrrolidone.

4. (canceled)

5. (canceled)

6. The method of claim 1, wherein the excipient comprises glycerin.

7. (canceled)

8. The method of claim 1, wherein the excipient comprises glycerin and polyvinylpyrrolidone.

9. (canceled)

10. (canceled)

11. The method of claim 1, wherein the preservative is selected from the group consisting of benzalkonium chloride, thimerosal, chlorobutanol, methyl paraben, propylparaben, phenyl ethyl alcohol, EDTA, sorbic acid, polyquarternium 1, and any combination thereof.

12. The method of claim 11, wherein the preservative has a concentration of about 0.001% to 1.0% by weight in the composition.

13. The method of claim 1, wherein the composition comprises a surfactant.

14. The method of claim 13, wherein the surfactant is selected from the group consisting of polysorbate 20, polysorbate 60, polysorbate 80, Pluronic F-68, Pluronic F-84, Pluronic F-127, Pluronic P-103, cyclodextrin, nonoxynol-9, Igepal Co-630, tyloxapol, and any combination thereof.

15. The method of claim 13, wherein the surfactant has a concentration of about 0.01% to 2% by weight in the composition.

16. The method of claim 1, wherein the demulcent comprises polyethylene glycol at a concentration of about 4% (w/w).

17. The method claim 1, wherein the composition has a pH of about 4.2 to 4.5.

18. The method of claim 1, wherein the composition is a viscous solution.

19. The method of claim 1, wherein the infectious conjunctivitis is bacterial conjunctivitis.

20. The method claim 1, wherein the infectious conjunctivitis is viral conjunctivitis.

21. A method for treating infectious conjunctivitis in a subject in need thereof, the method comprising administering to an infected eye of the subject a therapeutically effective amount of a composition consisting of:
about 0.6 to 1.25% (w/w) povidone-iodine;
about 1 to 2% (w/w) of an excipient selected from polyvinylpyrrolidone, glycerin, or a combination thereof;
optionally a preservative;
optionally a surfactant;
optionally a pH adjusting agent;
optionally a stabilizer; and
optionally a demulcent,
wherein the composition does not contain a steroidal anti-inflammatory drug or a non-steroidal anti-inflammatory drug.
